(19) United States
(12) Patent Application Publication
(10) Pub. No.: US 2023/0084476 A1
(43) Pub. Date: Mar. 16, 2023

(54) Urinary Output Measuring System

(71) Applicant: C. R. Bard, Inc., Franklin Lakes, NJ (US)

(72) Inventors: Jonathan Robichaud, Decatur, GA (US); Brett M. Eckles, Raleigh, NC (US)

(21) Appl. No.: 17/941,941

(22) Filed: Sep. 9, 2022

Related U.S. Application Data
(60) Provisional application No. 63/243,456, filed on Sep. 13, 2021.

(51) Int. Cl. A61B 5/20; A61M 25/00; A61B 5/00; A61F 5/44; B65B 69/00

(52) U.S. Cl. CPC A61B 5/208; A61M 25/0017; A61B 5/742; A61F 5/4404; B65B 69/0016

(57) ABSTRACT

Disclosed herein is a urinary output monitoring system. The system can include a urinary output monitoring device having a device body with a front panel and a back panel, and a fluid collection bag attachment configured to be detachably coupled to the front panel and to secure a fluid collection bag. The system can further include a load cell configured to determine a load placed upon the fluid collection bag attachment. The load cell can be in communication with a console. An attachment system can be configured to couple the urinary output monitoring system to a securing surface, for example to the back panel, and can have two or more contact points coupled to one or more rails defining a longitudinal axis, the contact points defining a lateral axis and configured to longitudinally move along the rails.

### PRIORITY

This application claims the benefit of priority to U.S. Provisional Application No. 63/243,456, filed Sep. 13, 2021, which is incorporated by reference in its entirety into this application.

### BACKGROUND

Some current urine monitoring systems use weight-based methodologies to determine urine volume and urine flow rate over time. A fluid collection container may be suspended from a weighing device and as urine is captured by the fluid collection container, the weight of the fluid collection container increases. The weighing device may be configured to determine the increase in the weight of the fluid collection container. However, most urine monitoring systems do not travel with a patient as the patient is moved through a hospital setting, requiring clinicians to suspend urine monitoring until a patient is stationed in one location. If a patient has to move frequently within the hospital (e.g., multiple surgeries), critical patient data may not be captured. It would be beneficial to the patient and the clinician to be able to monitor urine output during the patient's entire stay. Disclosed herein is a urinary output monitoring system and method of use that address the foregoing.

### SUMMARY

Disclosed herein is a urinary output monitoring device that includes a load cell coupled between a bag attachment member and a body of the urinary output monitoring device, where the load cell is configured to define an electrical signal based on a weight of urine output suspended from the bag attachment member. The device further includes an attachment system coupled with the body, where the attachment system is configured to secure the urinary output monitoring device to a securing surface. The device further includes a console coupled with the load cell, where the console includes a number of processors and a non-transitory computer readable storage medium having logic stored thereon that, when executed by the processors perform operations, and the operations include (i) receiving electrical signals from the load cell, (ii) converting the electrical signals into the weight data pertaining to the weight of urine output, (iii) converting the weight data into volume data, and (iv) transmitting the volume data to a display for depiction of the volume data thereon, where the display is wirelessly coupled with the urinary output monitoring device.

In some embodiments of the device, the bag attachment member is configured to couple with and suspend a urine collection bag, where the urine collection bag includes a drainage tube coupled with a urinary catheter so that urine output from patient is collected within the urine collection bag.

In some embodiments, the device further includes one or more sensors coupled with the console, where the one or more sensors are configured to determine the orientation the load cell, and the operations further include applying a correction factor to the weight data based on the orientation of the load cell.

In some embodiments of the device, the load cell includes a first load cell and a second load cell oriented at an angle with respect the first load cell, and the operations include (i) receiving a first electrical signal from the first load cell, (ii) receiving a second electrical signal from the second load cell, and (iii) converting a combination of the first electrical signal with the second electrical signal into the weight data.

In some embodiments of the device, the volume data includes a time day correlated therewith.

In some embodiments of the device, the operations further include receiving input from the display.

In some embodiments of the device, the urinary output monitoring device is located within a room of a medical facility during use, and the display is located outside the room during use.

In some embodiments of the device, the attachment system includes a first attachment mechanism configured to attach the urinary output monitoring device to at least one of a horizontal bar or a vertical bar and the attachment system includes a second attachment mechanism configured to attach the urinary output monitoring device to vertical flat surface.

In some embodiments of the device, the first attachment mechanism includes a clamp rotatably coupled with the body so that the clamp is rotatable between a horizontal orientation and a vertical orientation. In some embodiments of the device, the second attachment system includes a number of suction cups.

In some embodiments of the device, the display is a transportable computing device that includes at least one of a tablet, a cell phone, or a computer coupled with a medical cart.

Also disclosed herein is a urinary output monitoring system that includes a display and a urinary output monitoring device wirelessly coupled with the display. The urinary output monitoring device includes a load cell coupled between a bag attachment member and a body of the urinary output monitoring device, where the load cell is configured to define an electrical signal based on a weight of urine output suspended from the bag attachment member. The system further incudes an attachment system coupled with the body, where the attachment system is configured to secure the urinary output monitoring device to a securing surface. The system further incudes a console coupled with the load cell, where the console includes a number of processors and a non-transitory computer readable storage medium having logic stored thereon that, when executed by the processors perform operations. The operations include (i) receiving electrical signals from the load cell (ii) converting the electrical signals into the weight data pertaining to the weight of urine output, (iii) converting the weight data into volume data, and (iv) transmitting the volume data to the display for depiction of the volume data thereon.

In some embodiments, the system further includes a urine collection bag having a drainage tube configured for coupling with a urinary catheter, where the urine collection bag is configured for suspension from the bag attachment member.

In some embodiments of the system, the display defines a user interface of the urinary output monitoring system.

In some embodiments of the system, during use, the urinary output monitoring device is located within a room of a medical facility, and the display is located outside the room.

In some embodiments of the system, the attachment system includes a first attachment mechanism configured to attach the urinary output monitoring device to at least one of a horizontal bar or a vertical bar and a second attachment mechanism configured to attach the urinary output monitoring device to vertical flat surface.

Also disclosed herein is method of monitoring urine output from a patient. The method includes:
defining urine flow path between the patient and a urine collection bag, where the patient is located within a room a medical facility;
attaching a load cell to a securing surface;
suspending the urine collection bag from the load cell;
establishing a flow of urine along the urine flow path;
initiating a urine output monitoring process via a display wirelessly coupled with the load cell, where the display includes a user interface;
receiving an electrical signal from the load cell;
converting the electrical signal from the load cell into a volume of urine output collected within the urine collection bag;
depicting the volume of urine output on the display; and
observing the volume of urine output depicted on the display, where the display is positioned at a location outside the room.

In some embodiments, the method further includes transporting the display between a location within the room and the location outside the room during the urine output monitoring process.

In some embodiments of the method, attaching the load cell to a securing surface includes (i) attaching the load cell to a first securing surface during a first time period of the urine output monitoring process, where the first securing surface includes at least one of a horizontal bar or a vertical bar; (ii) attaching the load cell to a second securing surface during a second time period of the urine output monitoring process, where the second securing surface including a flat vertical surface.

In some embodiments, the method, further includes receiving an electrical signal from a sensor configured to determine an orientation of the load cell, and converting the electrical signal further includes applying a correction factor to the volume of urine output based on the electrical signal from the sensor.

These and other features of the concepts provided herein will become more apparent to those of skill in the art in view of the accompanying drawings and following description, which describe particular embodiments of such concepts in greater detail.

### DRAWINGS

A more particular description of the present disclosure will be rendered by reference to specific embodiments thereof that are illustrated in the appended drawings. It is appreciated that these drawings depict only typical embodiments of the invention and are therefore not to be considered limiting of its scope. Example embodiments of the invention will be described and explained with additional specificity and detail through the use of the accompanying drawings in which:

FIG. 1 illustrates a perspective view of an urinary output monitoring system, in accordance with some embodiments.

FIG. 2 illustrates a plan view of a back side of the urinary output monitoring system, in accordance with some embodiments.

FIG. 3 illustrates a block diagram of some components of the urinary output monitoring system including a console, in accordance with some embodiments.

FIGS. 4A-4E illustrates an exemplary method of coupling the urinary output monitoring system to a securing surface, in accordance with some embodiments.

FIG. 5 illustrates a flow chart of an exemplary method of coupling the urinary output monitoring system to the securing surface, in accordance with some embodiments.

FIG. 6A is a back view of another embodiment of the device of the system of FIG. 1, in accordance with some embodiments.

FIG. 6B is a left side view of device of FIG. 6A, in accordance with some embodiments.

FIG. 7 illustrates a flow chart of an exemplary method of monitoring urine output from a patient, in accordance with some embodiments.

### DESCRIPTION

Before some particular embodiments are disclosed in greater detail, it should be understood that the particular embodiments disclosed herein do not limit the scope of the concepts provided herein. It should also be understood that a particular embodiment disclosed herein can have features that can be readily separated from the particular embodiment and optionally combined with or substituted for features of any of a number of other embodiments disclosed herein.

Regarding terms used herein, it should also be understood the terms are for the purpose of describing some particular embodiments, and the terms do not limit the scope of the concepts provided herein. Ordinal numbers (e.g., first, second, third, etc.) are generally used to distinguish or identify different features or steps in a group of features or steps, and do not supply a serial or numerical limitation. For example, “first,” “second,” and “third” features or steps need not necessarily appear in that order, and the particular embodiments including such features or steps need not necessarily be limited to the three features or steps. Labels such as “left,” “right,” “top,” “bottom,” “front,” “back,” and the like are used for convenience and are not intended to imply, for example, any particular fixed location, orientation, or direction. Instead, such labels are used to reflect, for example, relative location, orientation, or directions. Singular forms of “a,” “an,” and “the” include plural references unless the context clearly dictates otherwise.

Unless defined otherwise, all technical and scientific terms used herein have the same meaning as commonly understood by those of ordinary skill in the art.

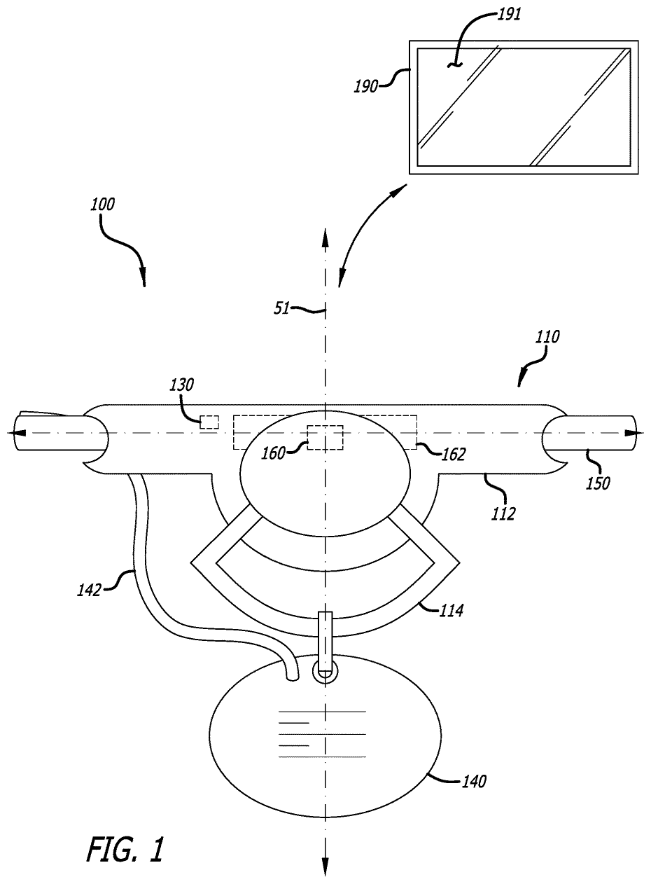

FIG. 1 illustrates a front view of a urinary output monitoring system 100, in accordance with some embodiments. The urinary output monitoring system (“system”) 100 is generally configured to monitor the urinary output of a patient over a defined time period, such as 1, 2, 3, or more days, for example. The system 100 is also configured to monitor the urine output while the patient undergoes various medical procedures which procedures may require the patient to be moved or relocated. As the patient is fluidly coupled with the system 100, the system 100 or a portion thereof may need to be relocated along with the patient. The system 100 is configured to (i) attach to a securing surface and (ii) weigh the urine output in a suspended container (e.g., bag). As such, in some instances, the system 100 or a portion thereof may need to be attached to a first securing surface (e.g., a bed rail) at a first location of the patient and subsequently attached to a second securing surface (e.g., a wall) at a second location of the patient.

The system 100 generally includes urinary output monitoring device (device) 110 and a urine collection bag (bag) 140. During use, the device 110 is attached to a securing surface 150 and the bag 140 is suspended from the device body 110. The bag 140 is selectively attachable to and detachable from the device 110. The device 112 is generally configured for multi-use across multiple patients and the bag 140 is generally configured for single-use across a single patient. In some instances, multiple bags 140 may be used for the same patient. The bag 140 includes drainage tube 142 coupleable with a urinary catheter (not shown), where the drainage tube 142 defines a urine flow path from the urinary catheter to the bag 140. In some embodiments, the fluid collection bag 140 may be fixedly attached to or otherwise include the urinary catheter.

The device 110 includes a device body 112 that generally defines a frame for the device 110 and a load cell 160 coupled between a bag attachment member 114 and the device body 112. The bag attachment member 114 may take any form suitable to enable the attachment of the bag 140 thereto in a suspended fashion. For example, the bag attachment member 114 may include a bar as illustrated, a hook, a ring, or a knob. The device body 112 may be coupled to the securing surface 150. More specifically, the device body 112 may be detachably coupled to the securing surface 150. In some instances of use, a clinician may attach and detach the device body 112 to and from the securing surface multiple times. The securing surface 150 may be any convenient surface suitable for attaching the device 110 thereto, such as a bed rail, an IV pole, or a wall, for example.

The device 110 includes a console 162 communicatively coupled with a display 190 so that system information thereon may be depicted on the display 190, where the system information includes urine output information or data. The display 150 is wirelessly coupled with the console 162 and physically separated from the device 110. Exemplary wireless communication modalities can include WiFi, Bluetooth, Near Field Communications (NFC), cellular Global System for Mobile Communication (“GSM”), electromagnetic (EM), radio frequency (RF), combinations thereof, or the like. The display 190 may be part of any suitable computing device, such as a personal computer, a network computer, a laptop, a tablet, or a cell phone, for example. The display 190 may be coupled with the console 162 via a local area network or the internet. The display 190 includes a user interface 191 (e.g., a graphical user interface) so that the clinician may interact with or otherwise operate the device 110 via the display 190.

The system 100 may be generally configured to minimize clinician interaction with the device 110. In some instances, clinician interaction may be limited to attaching the device 210 to the securing surface 150, hanging the bag 140 on the bag attachment member 114, removing the bag 140 from the bag attachment member 114, and detaching the device 210 from the securing surface 150. Other clinician interaction with the system 100 may occur via the display 190, where the display 190 is separated from the device 210. As such, aside from physical interaction, the clinician interact with the system 100 via the display 190 from a convenient location, such as an office, for example.

The load cell 160 is generally configured to (i) measure or otherwise determine load hangingly applied to the bag attachment member 114 and (ii) provide electrical signals to the console 162 in accordance with the load. In some embodiments, the device 110 may include more than one load cell 160. For example, the device 110 may include a first load cell aligned parallel with a transverse axis 51 the device body 112 and a second load cell aligned parallel with a longitudinal axis 52 (perpendicular to the transverse axis 51) of the device body 112. So arranged, the first and second load cells may account for an orientation of the device body 112, where the transverse axis 51 is not vertical. For example, in the case where the transverse axis 51 is vertical, the load may be determined by the first load sensor and in the case where the transverse axis 51 is horizontal and the longitudinal axis 52 is vertical, the load may be determined by the second load sensor. Further, in the case where the transverse axis 51 is oriented between the vertical and horizontal directions, the load may be determined by a combination of the first and second load sensors.

In some embodiments, the device 110 may include one or more sensors 130 configured to determine the orientation of the device body 112 (or more specifically the load cell 160), such as a gyroscope or an accelerometer, for example. In such embodiments, the one or more sensors 130 may provide an electrical signal to the console 162 indicating the orientation of the device body 112.

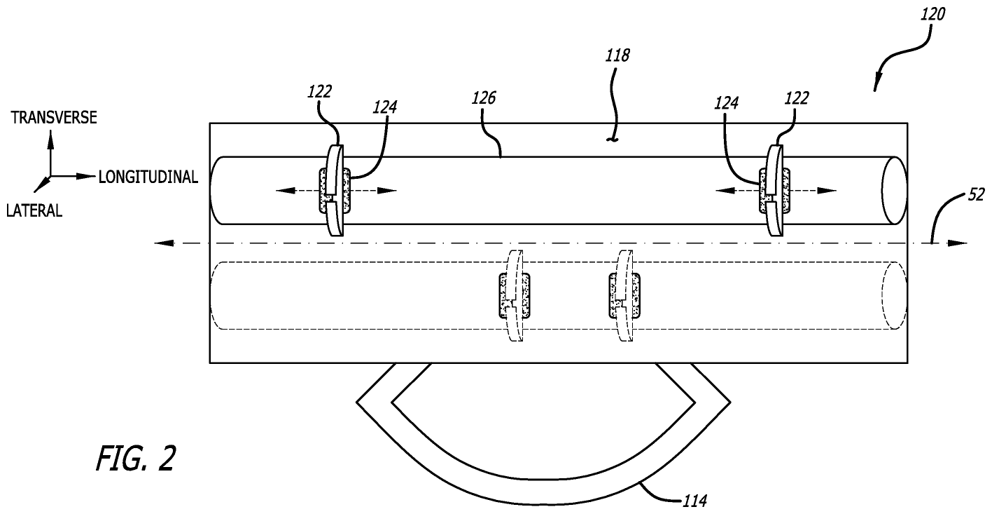

FIG. 2 illustrates a plan view of a back panel 118 of the device body 112, in accordance with some embodiments. In some embodiments, the back panel 118 may include an attachment system 120, wherein various attachments 122 may be used to couple the device body 112 to the securing surface 150. In some embodiments, various attachments 122 may be coupled to two or more contact points 124 configured to secure the device body 112 to the securing surface 150. In some embodiments, the two or more contact points 124 may define a lateral axis and receive the various attachments 122 thereon. In some embodiments, the various attachments 122 may be coupled to the two or more contact points 124 through a snap fit, an interference fit, a clip fit or the like. In some embodiments, each attachment 122 may be configured to rotate axially around the longitudinal axis or a lateral axis of each contact point 124. In some embodiments, each attachment 122 may be configured to independently rotate axially around the lateral axis of each contact point 124.

In some embodiments, the attachments 122 may include only one attachment that couples to each of the attachment points 124. In some embodiments, the attachment system 120 may include one or more rails 126 longitudinally coupled to the back panel 118 of the device body 112. In some embodiments, the one or more rails 126 may define the longitudinal axis 52. The two or more contact points 124 may be coupled to the one or more rails 126. Each contact point 124 of the two or more contact points 124 may be configured to independently slide longitudinally along the rails 126, allowing the device 112 to couple to any type of securing surface 150. The two or more contact points 124 may be configured to prevent the device body 112 from rotating around the longitudinal axis 194. In some instances, unnecessary rotation around the longitudinal axis 194 may impact the ability of the load cell 160 to accurately determine load cell values. The attachments 122 may include any suitable attachment mechanisms, such as hooks (e.g., J hooks, L hooks or the like), clamps, (e.g., circle clamps, spring clamps, pipe clamps, marman clamps, G/C clamps, locking clamps), clips, suction cups or the like. Advantageously, the attachment system 120 enable the urinary output monitoring device 110 to be coupled to any number of securing surfaces 150, allowing the urinary output monitoring system 100 to be mobile during use while consistently determining load cell values and moving with the user without interrupting use.

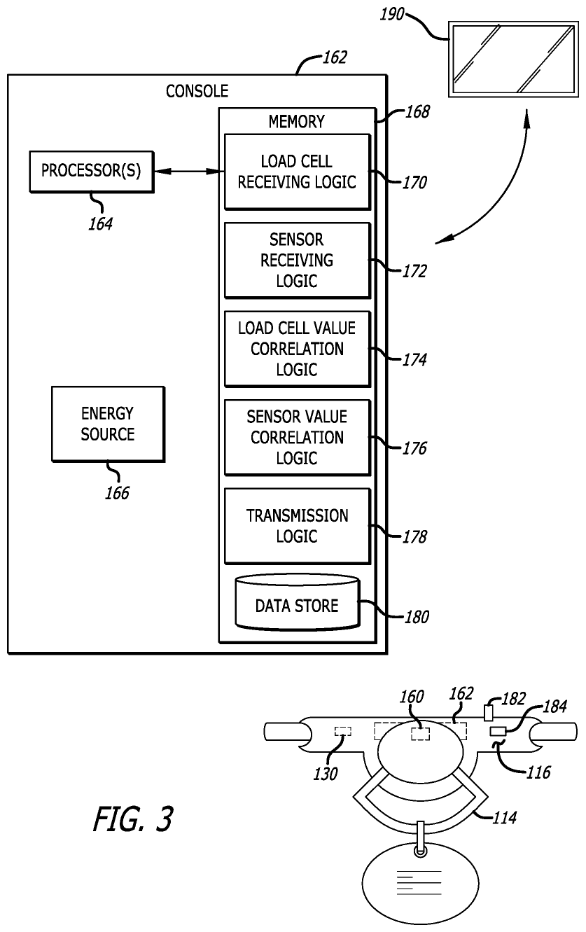

FIG. 3 illustrates a block diagram of some components of the urinary output monitoring system 100 including the console 162, in accordance with some embodiments. In some embodiments, the console 162 includes one or more processors 164, an energy source 166, non-transitory computer readable medium (“memory”) 168 and a plurality of logic modules. In some embodiments, the console 162 may include an actuator 182 (e.g., an on/off button or switch) coupled to the device 110. The console 162 is in wireless communication with the display 190. In some embodiments, the device 110 may also include a sync button 184 in communication with the console 162, configured to sync all the determined load cell measurements with the display 190. In some embodiments, the sync button 184 may be on a front panel 116 of the device body 112. In some embodiments, the load cell 160 may be wired to the console 162.

In some embodiments, the plurality of logic modules may include a load cell receiving logic 170, a sensor receiving logic 172, a load cell value correlation logic 174, a sensor value correlation logic 176, a transmission logic 178, and a data store 180. In some embodiments, the load cell receiving logic 170 may be configured to receive one or more load cell values or electrical signals from the load cell 160. In some embodiments the sensor receiving logic 172 may be configured to receive one or more sensor values or electrical signals from the one or more sensors 130 configured to determine the orientation of the device 110. In some embodiments, the load cell value correlation logic 174 may be configured to correlate the one or more load cell values with a volume value and a time of day value. In some embodiments, the volume value may correspond to the volume of fluid within the fluid collection bag 140 and the time of day value may correspond to the time of day the load cell value was determined. In some embodiments, the load cell value correlation logic 174 may be configured to correlate the load cell value with the volume value and time of day value as a {load cell value, volume value, time of day value}. In some embodiments, the sensor value correlation logic 176 may be configured to correlate the one or more sensor values with a degree value. In some embodiments, the degree value corresponds to the degree the orientation of the device 110 is away from level (e.g.,)0°. In some embodiments, the transmission logic 178 may be configured to transmit one or more of the load cell values, the volume values, the time of day values, the sensor values, or the degree values to the computing device or the electronic medical record system. In some embodiments, the transmission logic 178 may be configured to transmit one or more of the load cell values, the volume values, the time of day values, the sensor values, or the degree values to the display 190 as soon as the console 162 determines of the values or according to a user defined schedule (e.g., every 5 minutes, when a user presses the sync button, or the like). In some embodiments, the data store 180 may be configured to store one or more of the load cell values, the volume values, the time of day values, the sensor values, or the degree values until the console 162 communicates the values to the computing device or electronic medical record system.

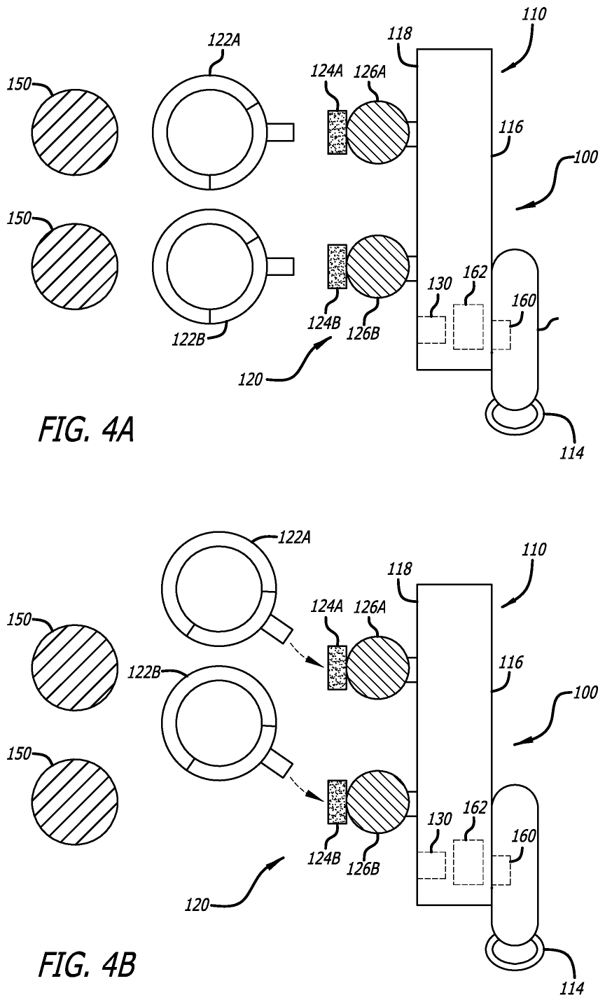

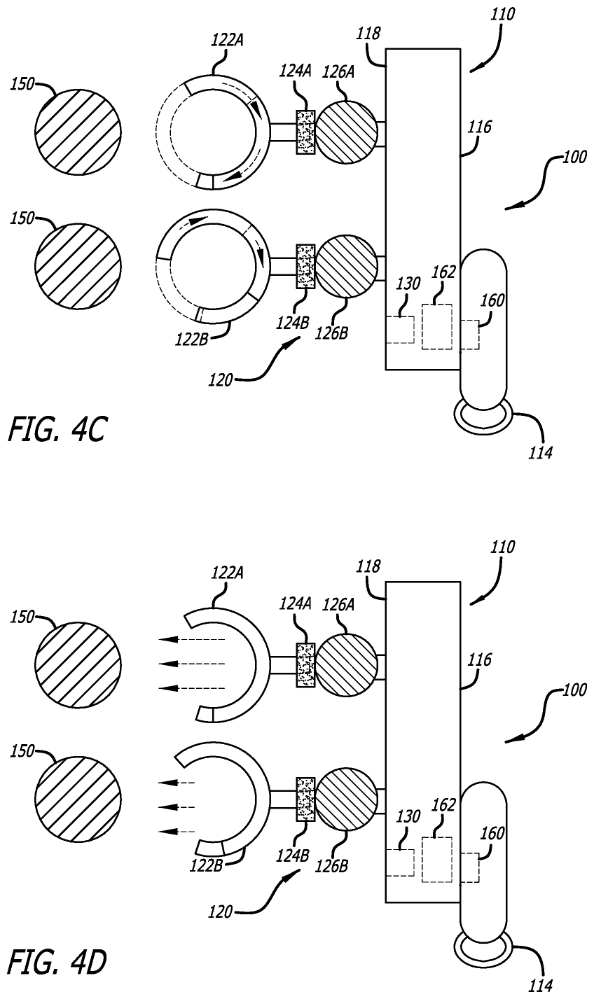

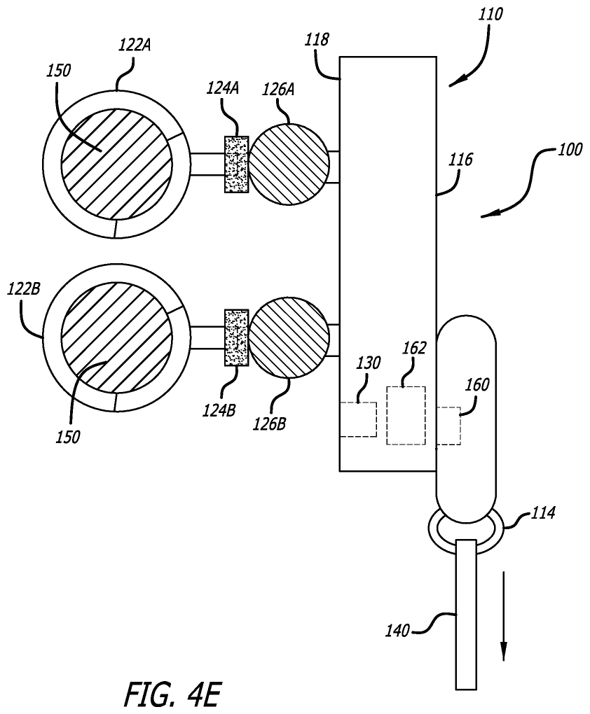

FIGS. 4A-4E illustrates a cross sectional side view of an exemplary method of coupling the urinary output monitoring system 100 to the securing surface 150, in accordance with some embodiments. In some embodiments, as illustrated in FIG. 4A, the urinary output monitoring device 110 may have the detachable fluid collection bag attachment 114 coupled to the front panel 116 and the attachment system 120 coupled to the back panel 118. In some embodiments, the attachments 122A-122B may be coupled to the two or more contact points 124A-124B, as illustrated in FIG. 4B. The attachments 122A-122B may be coupled to the two or more contact points 124A-124B through a snap fit, an interference fit, a press fit, or the like. Once the attachments 122A-122B are coupled to the two or more contact points 124A-124B, the two or more contact points 124 may be configured to be moved longitudinally on the two or more rails 126A-126B. The attachments 122A-122B may be configured to be transitioned from a closed configuration to an opened configuration, as illustrated in FIG. 4C.

The device 110 with the attachment system 120 including the attachments 122A-122B in the opened configuration may be configured to contact the securing surface 150 as illustrated in FIG. 4D. Once the attachments 122A-122B in the opened configuration contact the securing surface 150, the attachments 122A-122B may be transitioned to the closed configuration. Once the device 110 is coupled to the securing surface 150, the orientation of the device 110 may be determined by the one or more sensors 130 and the console 162, and the fluid collection bag 140 may be coupled to the detachable fluid collection bag attachment 114, as illustrated in FIG. 4E.

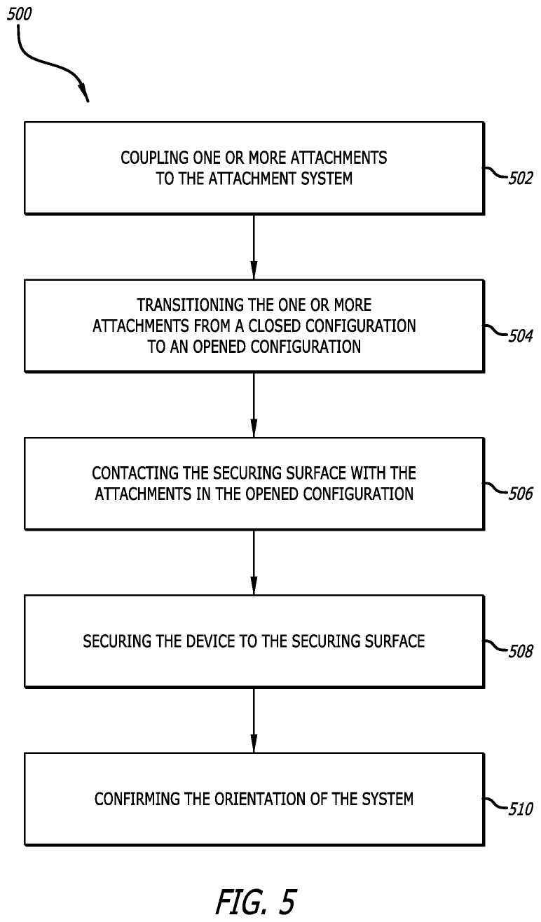

FIG. 5 illustrates a flow chart of an exemplary method 500 of coupling the urinary output monitoring system 100 to a securing surface 150. In some embodiments, the method 500 includes coupling one or more attachments 122 to the attachment system 120 (block 502). In some embodiments, coupling the one or more attachments 122 to the attachment system 120 includes coupling the one or more attachments 122 to the two or more contact points 124 that are coupled to the one or more rails 126. In some embodiments, the one or more attachments 122 may include hooks, clamps, clips, suction cups or the like.

The method 500 further includes transitioning the attachments 122 from a closed configuration to an opened configuration (block 504). In some embodiments, transitioning the attachments 122 from the closed configuration to the opened configuration may include sliding the attachments from the closed configuration to the opened configuration.

The method 500 further includes contacting the securing surface 150 with the attachments 122 in the opened configuration (block 506). In some embodiments, contacting the securing surface 150 includes slidably contacting the securing surface with the attachments 122.

The method 500 further includes securing the device 110 to the securing surface 150 (block 508). In some embodiments, securing the device 110 to the securing surface 150 includes transitioning the attachments 122 from the opened configuration to the closed configuration around the securing surface 150.

The method 500 further includes confirming the orientation of the system 100 (block 510). In some embodiments, confirming the orientation of the system 100 includes using the one or more sensors 130 and the console 162 to confirm the orientation of the system 100. In some embodiments, if the orientation of the system 100 is not optimal for the load cell 160 to determine load values, a user may adjust the attachments 122 on the securing surface 150 until the orientation of the system 100 is optimal for the load cell 160 to determine load values.

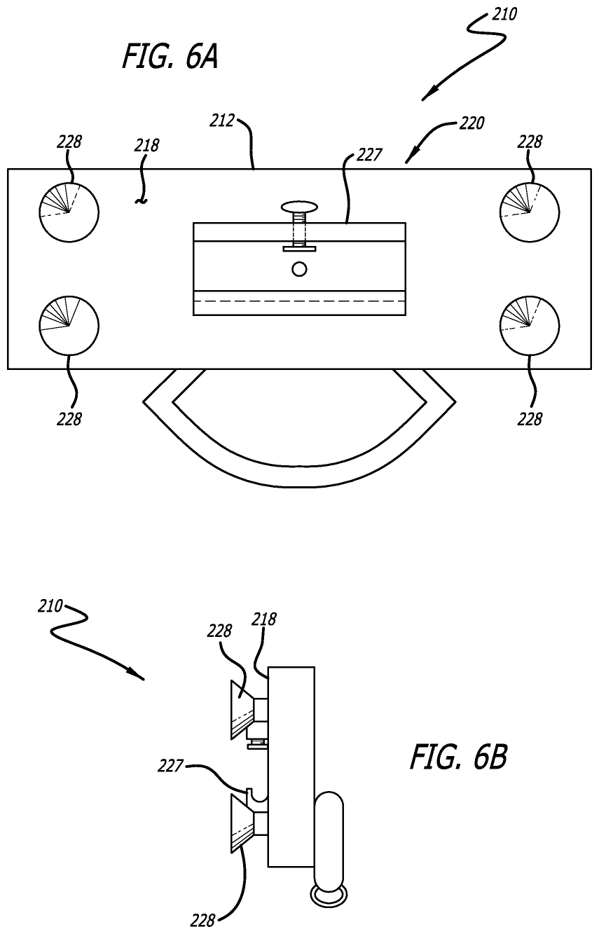

FIGS. 6A-6B illustrate another embodiment of a urinary output monitoring device 210 that can, in certain respects, resemble components of the urinary output monitoring device 110 described in connection with FIGS. 1-3. It will be appreciated that all the illustrated embodiments may have analogous features. Accordingly, like features are designated with like reference numerals, with the leading digits incremented to “2.” For instance, the device body is designated as “112” in FIGS. 1-3, and an analogous device body is designated as “212” in FIGS. 6A-6B. Relevant disclosure set forth above regarding similarly identified features thus may not be repeated hereafter. Moreover, specific features of the urinary output monitoring device 110 and related components shown in FIGS. 1-3 may not be shown or identified by a reference numeral in the drawings or specifically discussed in the written description that follows. However, such features may clearly be the same, or substantially the same, as features depicted in other embodiments and/or described with respect to such embodiments. Accordingly, the relevant descriptions of such features apply equally to the features of the urinary output monitoring device 210 of FIGS. 6A-6B. Any suitable combination of the features, and variations of the same, described with respect to the urinary output monitoring device 110 and components illustrated in FIGS. 1-3 can be employed with the urinary output monitoring device 210 and components of FIGS. 6A-6B, and vice versa.

FIG. 6A is a back view of the urinary output monitoring device (device) 210 and FIG. 6B is a left side view of the device 210. The device 210 includes an attachment system 220 having a number of different types of attachment mechanisms, where the different types of attachment mechanisms are configured to attach to different types of securing surfaces, such as a horizontal bar, a vertical bar, or a vertical flat surface, for example. As such, device 210 may be attached to a number of the different types of securing surfaces during use. For example, the clinician may attach the device 210 to a frame rail of bed during a first time period of a urine output monitoring process and attach the device 210 to a wall during a second time period of a urine output monitoring process.

In the illustrated embodiment, the attachment system 220 includes a clamp mechanism 227 coupled with the back panel 218 of the device body 212 where the clamp mechanism 227 is configured to attach the device 110 to an elongate bar, such as the frame rail of the bed for example. The clamp mechanism 227 is rotatably coupled with the device body 212. FIG. 6A shows the clamp mechanism 227 oriented for attachment to a horizontal bar. During use, the clamp mechanism 227 may be rotated with respect to the device body 212 so that the device 210 may be attached to a vertical bar, such as an IV pole, for example. In some embodiments, the attachment system 220 may include two clamp mechanisms rotatably fixed to the device body 212, where one clamp mechanism is attachable to a horizontal bar and the other clamp mechanism is attachable to a vertical bar.

The attachment system 220 further includes a number (e.g., 1, 2, 3, 4, or more) of suction cups 228 attach to the back panel 218 in combination with the clamp mechanism 227, where the suction cups 228 are configured to attach the device 210 to a vertical flat surface (e.g., a wall). During use, the clinician may employ either the claim mechanisms 227 or the suction cups 228 to secure the device 210 to the securing surface. Similarly, the clinician may employ both the claim mechanism 227 and the suction cups 228 during alternative time periods of the urine monitoring process.

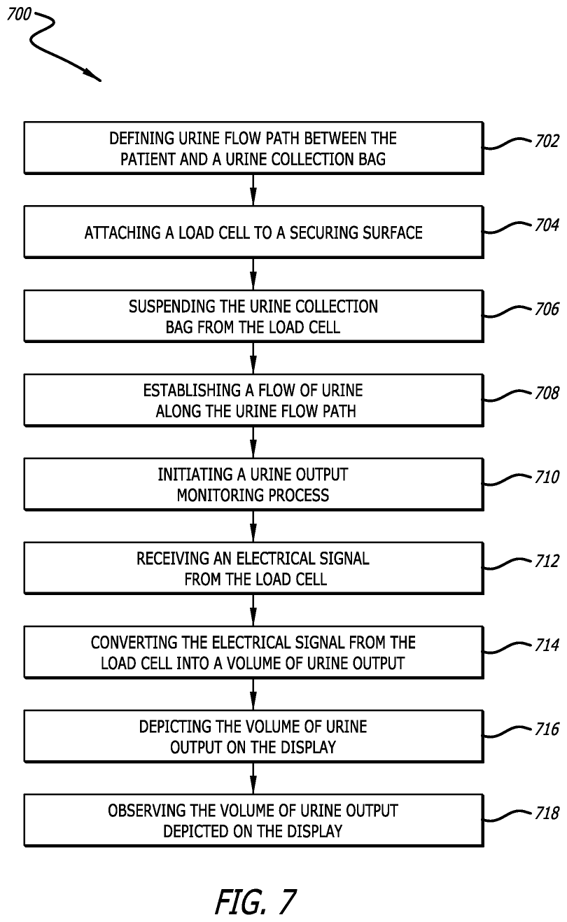

FIG. 7 illustrates a flow chart of a method of monitoring urine output from a patient where the method may include all or a subset of the following steps or processes, according to some embodiments.

The method may include defining urine flow path between the patient and a urine collection bag (block 702). The flow path may comprise the urinary catheter couple with a drainage tube that is further coupled with the urine collection bag so that urine flows from the patient to the urine collection bag.

The method may include attaching the load cell to the securing surface (block 704) where attachment the load cell includes attaching the device body to the securing surface so that the load cell is secured when the urine collection bag is suspended from the bag attachment member that coupled with the load cell. In some embodiments of the method, attaching the load cell to a securing surface may include attaching the load cell to a first securing surface such, as a bed rail or an IV pole, for example. At a later time, the clinician may attach the load cell to a second securing surface, such as a wall, for example.

The method may include suspending the urine collection bag from the load cell (block 706) so that the load cell may determine the weight of the urine collection having urine output collected therein.

The method may include establishing a flow of urine along the urine flow path (block 708) which may include catheterizing the patient or releasing a clamp on the urinary catheter.

The method may include initiating a urine output monitoring process (block 710) where initiating the urine output monitoring process include activating the load cell so that an electrical signal from the load cell relates to the weight of the urine collection bag having urine output collected therein. Initiating the urine output monitoring process may be performed via interaction with the display (user interface). The method may include receiving the electrical signal from the load cell (block 712) for processing by the logic.

The method may include converting the electrical signal from the load cell into a volume of urine output collected within the urine collection bag (block 714). Converting may include calculating a weight in accordance with a calibration of the load cell. Converting may further include calculating a volume of urine output from the weight based on a defined specific gravity of the urine.

Having calculated the volume of urine output, the method may include depicting the volume of urine output on the display (block 716) so that the clinician may observe the urine output volume (block 718) excreted by the patient over a period of time. The clinician may observe the urine output volume as depicted on the display (block 718) at the current location of the display which may be outside of the room where in the patient resides.

In some embodiments, the method may further include transporting the display between a location within the room and the location outside the room during the urine output monitoring process. For example, the display may be a portable device, such as a tablet, for example and the clinician may carry the display to different areas of the medical facility.

In some embodiments, the method further include receiving an electrical signal from the one or more sensors configured to determine an orientation of the load cell, and the logic may apply a correction factor to the volume of urine output based on the electrical signal as part of converting the electrical signal to a volume of urine output.

While some particular embodiments have been disclosed herein, and while the particular embodiments have been disclosed in some detail, it is not the intention for the particular embodiments to limit the scope of the concepts provided herein. Additional adaptations and/or modifications can appear to those of ordinary skill in the art, and, in broader aspects, these adaptations and/or modifications are encompassed as well. Accordingly, departures may be made from the particular embodiments disclosed herein without departing from the scope of the concepts provided herein.

What is claimed is:

1. A urinary output monitoring device, comprising:
a load cell coupled between a bag attachment member and a body of the urinary output monitoring device, the load cell configured to define an electrical signal based on a weight of urine output suspended from the bag attachment member;
an attachment system coupled with the body, the attachment system configured to secure the urinary output monitoring device to a securing surface;
a console coupled with the load cell, the console including a number of processors and a non-transitory computer readable storage medium having logic stored thereon that, when executed by the processors perform operations, including:
receiving electrical signals from the load cell;
converting the electrical signals into the weight data pertaining to the weight of urine output;
converting the weight data into volume data; and
transmitting the volume data to a display for depiction of the volume data thereon, the display wirelessly coupled with the urinary output monitoring device.

2. The device according to claim 1, wherein the bag attachment member is configured to couple with and suspend a urine collection bag, the urine collection bag including a drainage tube coupled with a urinary catheter so that urine output from patient is collected within the urine collection bag.

3. The device according to claim 1, further comprising one or more sensors coupled with the console, the one or more sensors configured to determine the orientation the load cell, wherein the operations further include applying a correction factor to the weight data based on the orientation of the load cell.

4. The device according to claim 1, wherein:
the load cell includes a first load cell and a second load cell oriented at an angle with respect the first load cell, and
the operations include:
receiving a first electrical signal from the first load cell;
receiving a second electrical signal from the second load cell; and
converting a combination of the first electrical signal with the second electrical signal into the weight data.

5. The device according to claim 1, wherein the volume data includes a time day correlated therewith.

6. The device according to claim 1, wherein the operations further include receiving input from the display.

7. The device according to claim 1, wherein, during use:
the urinary output monitoring device is located within a room of a medical facility, and
the display is located outside the room.

8. The device according to claim 1, wherein the attachment system includes:
a first attachment mechanism configured to attach the urinary output monitoring device to at least one of a horizontal bar or a vertical bar; and
a second attachment mechanism configured to attach the urinary output monitoring device to vertical flat surface.

9. The device according to claim 1, wherein the first attachment mechanism includes a clamp rotatably coupled with the body so that the clamp is rotatable between a horizontal orientation and a vertical orientation.

10. The device according to claim 1, wherein the second attachment system includes a number of suction cups.

11. The device according to claim 1, wherein the display is a transportable computing device, including at least one of a tablet, a cell phone, or a computer coupled with a medical cart.

12. A urinary output monitoring system, comprising:
a display;
a urinary output monitoring device wirelessly coupled with the display, the urinary output monitoring device, comprising:
a load cell coupled between a bag attachment member and a body of the urinary output monitoring device, the load cell configured to define an electrical signal based on a weight of urine output suspended from the bag attachment member;
an attachment system coupled with the body, the attachment system configured to secure the urinary output monitoring device to a securing surface;
a console coupled with the load cell, the console including a number of processors and a non-transitory computer readable storage medium having logic stored thereon that, when executed by the processors perform operations, including:
receiving electrical signals from the load cell;
converting the electrical signals into the weight data pertaining to the weight of urine output;
converting the weight data into volume data; and
transmitting the volume data to the display for depiction of the volume data thereon.

13. The system according to claim 12, further comprising a urine collection bag having a drainage tube configured for coupling with a urinary catheter, the urine collection bag configured for suspension from the bag attachment member.

14. The system according to claim 12, wherein the display defines a user interface of the urinary output monitoring system.

15. The system according to claim 12, wherein, during use:
the urinary output monitoring device is located within a room of a medical facility, and the display is located outside the room.

16. The system according to claim 12, wherein the attachment system includes:
a first attachment mechanism configured to attach the urinary output monitoring device to at least one of a horizontal bar or a vertical bar; and
a second attachment mechanism configured to attach the urinary output monitoring device to vertical flat surface.

17. A method of monitoring urine output from a patient, comprising:
defining urine flow path between the patient and a urine collection bag, the patient located within a room a medical facility;
attaching a load cell to a securing surface;
suspending the urine collection bag from the load cell;
establishing a flow of urine along the urine flow path;
initiating a urine output monitoring process via a display wirelessly coupled with the load cell, the display including a user interface;
receiving an electrical signal from the load cell;
converting the electrical signal from the load cell into a volume of urine output collected within the urine collection bag;
depicting the volume of urine output on the display; and
observing the volume of urine output depicted on the display, the display positioned at a location outside the room.

18. The method according to claim 17, further comprising transporting the display between a location within the room and the location outside the room during the urine output monitoring process.

19. The method according to claim 17, wherein attaching the load cell to a securing surface includes:
attaching the load cell to a first securing surface during a first time period of the urine output monitoring process, the first securing surface including at least one of a horizontal bar or a vertical bar; and
attaching the load cell to a second securing surface during a second time period of the urine output monitoring process, the second securing surface including a flat vertical surface.

20. The method according to claim 17, further comprising receiving an electrical signal from a sensor configured to determine an orientation of the load cell,
wherein converting the electrical signal further includes applying a correction factor to the volume of urine output based on the electrical signal from the sensor.
